(19) United States
(12) Patent Application Publication
(10) Pub. No.: US 2024/0091032 A1
(43) Pub. Date: Mar. 21, 2024

(54) Prosthetic Sock with Distal Fabric Reinforcement

(71) Applicants: Aldo Laghi, Pinellas Park (CZ); AIps South Europe, S. R.O, Pizen (CZ)

(72) Inventors: Aldo Laghi, Pinellas Park, FL (US); Nathaniel Vint, Palm Harbor, FL (US)

(21) Appl. No.: 18/234,664

(22) Filed: Aug. 16, 2023

Related U.S. Application Data
(60) Provisional application No. 63/408,202, filed on Sep. 20, 2022.

(51) Int. Cl. A61F 2/78

(52) U.S. Cl. CPC A61F 2/7812; A61F 2002/7818

(57) ABSTRACT

A sock comprising an outer fabric layer that tapers from a proximal end down to a distal end. The distal end of the sock further comprises a fabric disc which can be mechanically or chemically applied to the outer fabric layer. The distal end of the sock further comprises a channel which allows for the receipt of a prosthetic (not illustrated). Likewise, the fabric disc comprises a complementary disc channel which allows for the receipt of a prosthetic. No glue is present on the interior rim of the channel or the interior disc rim of the disc channel. Preferably the fabric disc is made of polyester but may be made of any material that has a horizontal stretch that is at least 20% less than that of the exterior fabric layer. Further, the fabric disc preferably has an outer diameter between ½ and 2 inches and an inner diameter between ¼ to ½ inches. The fabric disc is attached to the distal end using a heat activated polyurethane adhesive layer but may be applied using fabric glue as well.

### CROSS-REFERENCE TO RELATED APPLICATIONS

This application claims priority to U.S. Provisional Application No. 63/408,202, filed Sep. 20, 2022, the contents of which are incorporated by reference herein in their entirety.

### BACKGROUND OF THE INVENTION

### Field of the Invention

The described invention relates to socks for use in a prosthetic assembly. Specifically, the described invention relates to prosthetic socks having a fabric disc applied to a distal end so as to reinforce and strengthen the distal end of the prosthetic sock or liner. Alternatively, the invention may apply to prosthetic liners as well.

### Description of the Background Art

A residual limb normally experiences volume reductions over time which can have an outsized effect on the fit of an artificial prosthesis. Prosthetic socks are generally knitted pieces of fabric that serve to shore up the fit between a prosthetic liner and a prosthesis and can have varying thickness. These prosthetic socks generally need a channel for the receipt of the prosthesis and, due to their knitted nature, can have loose threads around that channel. These loose threads sometimes end up going into the lock when the patient engages the prosthesis causing the lock to become jammed. Previous attempts to solve this issue included gluing around the open channel in the sock but the glue would become hard and bulky upon drying thereby causing patient discomfort. This can also create distal thickness stacking which could create up to a quarter of an inch growth which can push the patient out of a socket which is uncomfortable.

Therefore, it is an object of this invention to provide an improvement which overcomes the aforementioned inadequacies of the prior art devices and provides an improvement which is a significant contribution to the advancement of the prosthetic sock art.

Another objective of the invention is to create a strengthened distal end of a prosthetic sock that allows for engagement with a prosthetic lock without jamming.

Another objective of the invention is to not require the use of glue around the interior rim of the channel of the prosthetic sock.

The foregoing has outlined some of the pertinent objects of the invention. These objects should be construed to be merely illustrative of some of the more prominent features and applications of the intended invention. Many other beneficial results can be attained by applying the disclosed invention in a different manner or modifying the invention within the scope of the disclosure. Accordingly, other objects and a fuller understanding of the invention may be had by referring to the summary of the invention and the detailed description of the preferred embodiment in addition to the scope of the invention defined by the claims taken in conjunction with the accompanying drawings.

### SUMMARY OF THE INVENTION

The present invention relates generally to a sock comprising an outer fabric layer that tapers from a proximal end down to a distal end. The distal end of the sock further comprises a fabric disc which can be mechanically or chemically applied to the outer fabric layer. The distal end of the sock further comprises a channel which allows for the receipt of a prosthetic (not illustrated). Likewise, the fabric disc comprises a complementary disc channel which allows for the receipt of a prosthetic. No glue is present on the interior rim of the channel or the interior disc rim of the disc channel. Preferably the fabric disc is made of polyester but may be made of any material that has a horizontal stretch that is at least 20% less than that of the exterior fabric layer. Further, the fabric disc preferably has an outer diameter between 0.5 and 4 inches and an inner diameter between 0.25 to 1 inches. The fabric disc is attached to the distal end using a heat activated polyurethane adhesive layer but may be applied using fabric glue as well.

The foregoing has outlined rather broadly the more pertinent and important features of the present invention in order that the detailed description of the invention that follows may be better understood so that the present contribution to the art can be more fully appreciated. Additional features of the invention will be described hereinafter which form the subject of the claims of the invention. It should be appreciated by those skilled in the art that the conception and the specific embodiment disclosed may be readily utilized as a basis for modifying or designing other structures for carrying out the same purposes of the present invention. It should also be realized by those skilled in the art that such equivalent constructions do not depart from the spirit and scope of the invention as set forth in the appended claims.

### BRIEF DESCRIPTION OF THE DRAWINGS

For a more complete understanding of the present disclosure and its advantages, reference is now made to the following descriptions, taken in conjunction with the accompanying drawings, in which:

FIG. 1 is a perspective view of a sock implementing the fabric reinforcement;

FIG. 2 is a cross-sectional view along line H-H of a sock implementing the fabric reinforcement;

FIG. 2A is a magnified view of Detail A showing the relationship between the sock and the fabric reinforcement;

FIG. 3 is a perspective view of a prosthetic liner implementing the fabric reinforcement;

FIG. 4 is a cross-sectional view along line J-J of a prosthetic liner implementing the fabric reinforcement; and

FIG. 4A is a magnified view of Detail K showing the relationship between the prosthetic liner and the fabric reinforcement.

Similar reference numerals refer to similar parts throughout the several views of the drawings.

### DETAILED DESCRIPTION OF THE DRAWINGS

The following description is of the best mode presently contemplated for carrying out the invention. This description is not to be taken in a limiting sense, but is made merely for the purpose of describing one or more preferred embodiments of the invention. The scope of the invention should be determined with reference to the claims.

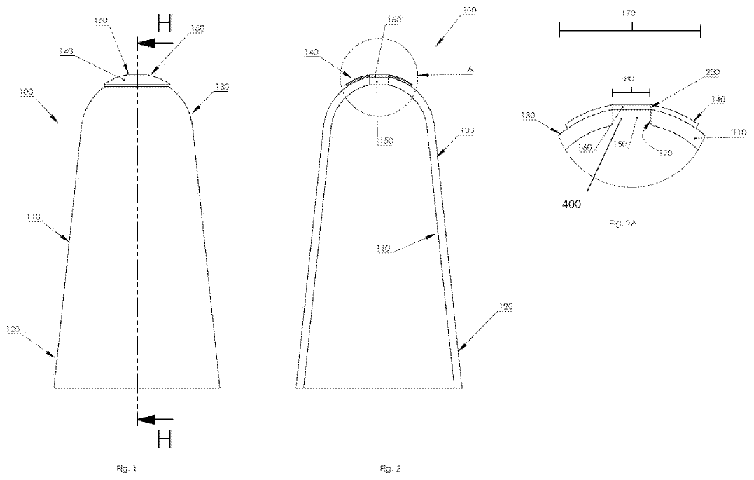

FIGS. 1-2A illustrate a preferred embodiment of a sock 100 comprising an outer fabric layer 110 that tapers from a proximal end 120 down to a distal end 130. The distal end 130 of the sock 100 further comprises a fabric disc 140 which can be mechanically or chemically applied to the outer fabric layer 110. The distal end 130 of the sock 100 further comprises a centrally placed channel 150 which allows for the receipt of a prosthetic (not illustrated). Likewise, the fabric disc 140 comprises a complementary disc channel 160 which allows for the receipt of a prosthetic through the creation of a central channel 400. No glue is present on the interior rim 190 of the central channel 400 or the interior disc rim 200 of the disc channel 160.

Preferably the fabric disc 140 is made of polyester but may be made of any material that has a horizontal stretch that is at least 20% less than that of the outer fabric layer 110. For example, if the outer fabric layer 110 stretches five inches at the proximal end, the fabric disc 140 should only be able to stretch four inches. In terms of shape, the fabric disc 140 and disc channel 160 are preferably circular but may be any shape that allows for the receipt of a prosthesis, such as square or oval-shaped. When the fabric disc 140 is circular in shape, the fabric disc 140 preferably has an outer diameter 170 between 0.5 and 4 inches and an inner diameter 180 between 0.25 to 1 inches. This inner diameter 180 is the same for the central channel 400. The fabric disc 140 is attached to the distal end 130 using a heat activated polyurethane adhesive layer but may be applied using fabric glue as well.

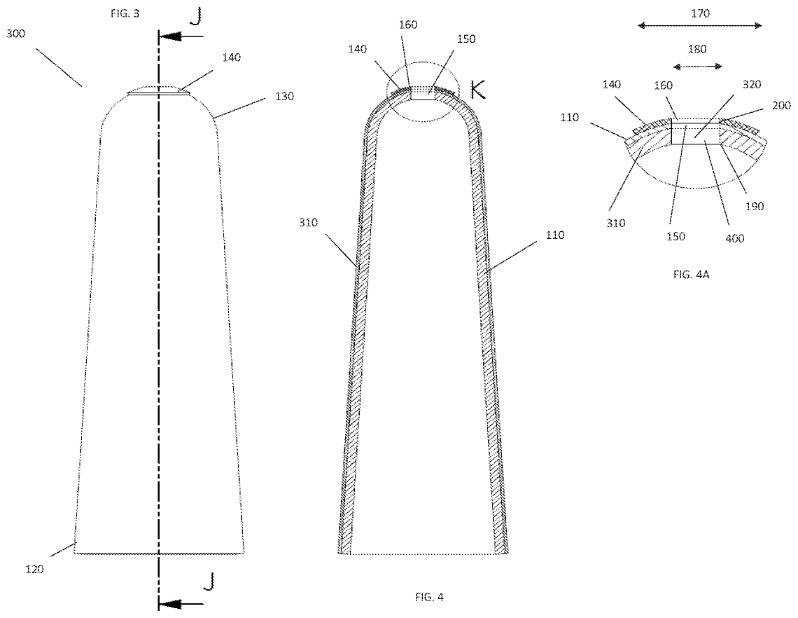

Alternatively, as shown in FIGS. 3-4A, the present invention may be used in combination with a prosthetic liner 300. The prosthetic liner 300 may further comprise an inner elastomer layer 310 comprising either silicone, urethane, or a thermoplastic elastomer such as poly(styrene-ethylene-butylene-styrene), i.e. SEEPS, or any other linear triblock copolymers that are capable of making a solid gel composition. In all other regards, the fabric disc 140 is applied in the same manner as described above with the sock 100 with the addition of an elastomer channel 320 for the insertion of a prosthetic lock or other prosthetic device. The elastomer channel 320 can also act as a moisture port/expulsion zone to allow fluid built up between the inner elastomer layer 310 and the user's limb to be expelled out of the elastomer channel 320.

The present disclosure includes that contained in the appended claims, as well as that of the foregoing description. Although this invention has been described in its preferred form with a certain degree of particularity, it is understood that the present disclosure of the preferred form has been made only by way of example and that numerous changes in the details of construction and the combination and arrangement of parts may be resorted to without departing from the spirit and scope of the invention.

Now that the invention has been described,

What is claimed is:

1. A reinforced prosthetic sock comprising:
an outer fabric layer having a closed distal end and an open proximal end wherein the closed distal end further comprises a centrally located channel having an interior fabric rim; and
a fabric disc comprising a disc channel, an interior disc rim, and an outer diameter between 0.5 and 4 inches, wherein the fabric disc further comprises polyester, has a horizontal stretch 20% less than the outer fabric layer, and is circular, the fabric disc being glued to the closed distal end of the outer fabric layer such that the centrally located channel and the disc channel create a concentric central channel, the central channel having an inner diameter between 0.25 and 1 inches and wherein the central channel, interior disc rim, and interior fabric rim are devoid of glue.

2. A reinforced prosthetic sock comprising:
an outer fabric layer having a closed distal end and an open proximal end wherein the closed distal end further comprises a centrally located channel; and
a fabric disc comprising a disc channel and an interior disc rim, wherein the fabric disc is attached to the closed distal end such that the centrally located channel and the disc channel create a concentric central channel.

3. The reinforced prosthetic sock of claim 2 wherein the fabric disc further comprises polyester.

4. The reinforced prosthetic sock of claim 2 wherein the fabric disc is circular.

5. The reinforced prosthetic sock of claim 4 wherein the fabric disc has an outer diameter between 0.5 and 4 inches.

6. The reinforced prosthetic sock of claim 4 wherein the central channel has an inner diameter between 0.25 and 1 inches.

7. The reinforced prosthetic sock of claim 2 wherein the fabric disc is glued to the closed distal end of the outer fabric layer such that the centrally located channel and the disc channel create a concentric central channel.

8. The reinforced prosthetic sock of claim 7 wherein the central channel is devoid of glue.

9. The reinforced prosthetic sock of claim 2 wherein the fabric disc is attached to the closed distal end of the outer fabric layer with a heat activated polyurethane adhesive.

10. The reinforced prosthetic sock of claim 2 wherein the fabric disc has a horizontal stretch 20% less than the outer fabric layer.

11. A reinforced prosthetic sock comprising:
an outer fabric layer having a closed distal end and an open proximal end wherein the closed distal end further comprises a centrally located channel; and
a fabric disc comprising a disc channel and an interior disc rim, wherein the fabric disc is attached to the closed distal end with a heat activated polyurethane adhesive and wherein the fabric disc is attached such that the centrally located channel and the disc channel create a concentric central channel.

12. The reinforced prosthetic sock of claim 11 wherein the fabric disc is circular.

13. The reinforced prosthetic sock of claim 12 wherein the fabric disc has an outer diameter between 0.5 and 4 inches.

14. The reinforced prosthetic sock of claim 12 wherein the fabric disc has an inner diameter between 0.25 and 1 inches.

15. The reinforced prosthetic sock of claim 11 wherein the fabric disc further comprises polyester.

16. The reinforced prosthetic sock of claim 11 wherein the fabric disc has a horizontal stretch 20% less than the outer fabric layer.
